(19) United States
(12) Patent Application Publication
(10) Pub. No.: US 2026/0265162 A1
(43) Pub. Date: Sep. 10, 2026

(54) MULTI-STEP DEFLUORINATION OF MIXTURES OF FLUORINATED COMPOUNDS

(71) Applicant: INTERNATIONAL BUSINESS MACHINES CORPORATION, ARMONK, NY (US)

(72) Inventors: Pedro Arrechea, San Jose, CA (US); James L. Hedrick, Pleasanton, CA (US); Nathaniel H. Park, San Jose, CA (US); Benjamin Hardy Wunsch, Mt. Kisco, NY (US); Ishwar Singh, Sunnyvale, CA (US)

(21) Appl. No.: 19/072,054

(22) Filed: Mar. 6, 2025

(51) Int. Cl. C07C 1/30; B01D 53/86; B01D 53/88

(52) U.S. Cl. CPC C07C 1/30; B01D 53/8662; B01D 53/88; B01D 2255/70; B01D 2255/802; B01D 2257/204; B01D 2257/2066; B01D 2258/0216

(57) ABSTRACT

A process includes receiving, at a first reactor from a set of reactors, a mixture of fluorinated compounds. The process also includes partially defluorinating the mixture of fluorinated compounds by a first defluorinating species on a solid support and substantially defluorinating, by at least one additional defluorinating species in at least one additional reactor, the partially defluorinated mixture. A system includes a set of reactors configured to operate in sequence to substantially defluorinate a mixture of fluorinated compounds. The operating in sequence comprises receiving the mixture at a first reactor, defluorinating, by a first defluorinating species, a first species of the fluorinated compounds, and defluorinating, by a second defluorinating species in a second reactor, a second species of the fluorinated compounds.

### BACKGROUND

The present disclosure relates to reactions of halogenated compounds and, more specifically, to destruction and sequestration of fluorinated compounds.

Fluorinated compounds, such as perfluorocarbons, per- and polyfluoroalkanes, sulfur hexafluoride, etc., have a wide number of applications. In semiconductor manufacturing, fluorinated and other process gases provide selectivity and specificity in processes such as etching, deposition, and chamber cleaning. However, the exhaust gas from these processes must be scrubbed of unreacted or partially reacted fluorinated compounds before being released into the environment. Fluorinated compounds such as per- and polyfluoroalkyl substances (PFAS) are used in many consumer products due to their unique properties associated with carbon-fluorine bonds. However, because of the inertness of these bonds, PFAS are slow to degrade in the environment. Concerns about the safety and bioavailability of fluorinated compounds have led to the development of various techniques for abatement of these compounds.

### SUMMARY

Various embodiments are directed to a process that includes receiving, at a first reactor from a set of reactors, a mixture of fluorinated compounds. The process also includes partially defluorinating, by a first defluorinating species in the first reactor, the mixture of fluorinated compounds, wherein the first defluorinating species is a solid supported species. Further, the process includes substantially defluorinating, by at least one additional defluorinating species in at least one additional reactor from the set of reactors, the partially defluorinated mixture of fluorinated compounds.

Additional embodiments are directed to a system that includes a first reactor, which is configured to receive a mixture of fluorinated compounds and partially defluorinate, by a first defluorinating species, the mixture of fluorinated compounds. The first defluorinating species is a solid supported species. The system also includes at least one additional reactor, which is configured to substantially defluorinate, by at least one additional defluorinating species, the partially defluorinated mixture of fluorinated compounds.

Further embodiments are directed to a system that includes a set of reactors configured to operate in sequence to substantially defluorinate a mixture of fluorinated compounds and a process that includes substantially defluorinating, by a set of reactors configured to operate in sequence, a mixture of fluorinated compounds. The operating in sequence includes receiving, at a first reactor from the set of reactors, the mixture of fluorinated compounds. The operating in sequence also includes defluorinating, by a first defluorinating species in the first reactor, a first species of fluorinated compound in the mixture of fluorinated compounds and defluorinating, by a second defluorinating species in a second reactor from the set of reactors, a second species in the mixture of fluorinated compounds.

Additional embodiments are directed to a system that includes a set of reactors configured to operate in sequence to substantially defluorinate a mixture of fluorinated compounds. The set of reactors includes a reactor containing a first defluorinating species on a solid support. The first defluorinating species is an alkali metal, and the solid support is an oxide.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a flow diagram illustrating a process of defluorination, according to some embodiments.

FIG. 2 is a block diagram illustrating a system for defluorination, according to some embodiments.

FIG. 3A-3H are schematic diagrams illustrating defluorination reaction environments, according to some embodiments.

While the invention is amenable to various modifications and alternative forms, specifics thereof have been shown by way of example in the drawings, and will be described in detail. It should be understood, however, that the intention is not to limit the invention to the particular embodiments described. Instead, the intention is to cover all modifications, equivalents, and alternatives falling within the spirit and scope of the invention.

### DETAILED DESCRIPTION

Embodiments of the present invention are generally directed to reactions of halogenated compounds and, more specifically, to abatement of fluorinated compounds. While the present disclosure is not necessarily limited to such applications, various aspects of the disclosure may be appreciated through a discussion of examples using this context.

Although the present invention has been described in reference to specific embodiments, it should be understood that the invention is not limited to these examples only and that many variations of these embodiments may be readily envisioned by the skilled person after having read the present disclosure. The invention may thus further be described without limitation, and by way of example only, by the following clauses.

Clause 1. A process, comprising: receiving, at a first reactor from a set of reactors, a mixture of fluorinated compounds; partially defluorinating, by a first defluorinating species in the first reactor, the mixture of fluorinated compounds, wherein the first defluorinating species is a solid supported species; and substantially defluorinating, by at least one additional defluorinating species in at least one additional reactor from the set of reactors, the partially defluorinated mixture of fluorinated compounds. A technical effect of this process can be removing fluorinated compounds from a mixture of compounds.

Clause 2. The process of clause 1, wherein the partially defluorinating the mixture of fluorinated compounds comprises partially defluorinating a first species of fluorinated compound in the mixture of fluorinated compounds. This can have the technical effect of enabling subsequent defluorination reactions with the partially defluorinated first species.

Clause 3. The process of clause 2, wherein the substantially defluorinating the mixture of fluorinated compounds comprises completely defluorinating the partially defluorinated first species of fluorinated compound. This can have the technical effect of substantially removing the first species from the mixture of compounds.

Clause 4. The process of any of clauses 1-3, wherein the partially defluorinating the mixture of fluorinated compounds comprises completely defluorinating a first species of fluorinated compound in the mixture of fluorinated compounds. This can have the technical effect of substantially removing the first species from the mixture of compounds.

Clause 5. The process of any of clauses 1-4, wherein the first defluorinating species is a photocatalyst. This can have the technical effect of defluorinating compounds via photocatalyzed reaction.

Clause 6. The process of clause 5, wherein the photocatalyst is selected from the group consisting of [4,4′-bis(1,1-dimethylethyl)-2,2′-bipyridine-N1,N1′]bis[2-(2-pyridinyl-N)phenyl-C]iridium(III) hexafluorophosphate, tris(2,2′-bipyridine)ruthenium(II) hexafluorophosphate, N-phenylphenothiazine, 1,2,3,5-tetrakis(carbazol-9-yl)-4,6-dicyanobenzene, and [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate. This can have the technical effect of enabling photocatalyzed reactions with one or more fluorinated species.

Clause 7. The process of any of clauses 1-4, wherein the first defluorinating species is an alkali metal absorbed by an oxide. This can have the technical effect of defluorinating compounds via reactions with the alkali metal. An advantage of the absorption by the oxide can be that it allows safer handling of the alkali metal.

Clause 8. The process of any of clauses 1-4 wherein the first defluorinating species is selected from the group consisting of a palladium hydrogenation catalyst, a photocatalyst, a sequestration polymer, and an alkali metal. The palladium hydrogenation catalyst, photocatalyst, and alkali metal can have the technical effect of defluorinating fluorinated compounds, and the sequestration polymer can have the technical effect of sequestering fluorinated compounds.

Clause 9. The process of clause 8, wherein: the at least one additional reactor is a photoreactor; and the at least one additional defluorinating species comprises hydrated electrons. The technical effect of the photoreactor can be generating the hydrated electrons in an aqueous solution. The hydrated electrons may then hydrogenate fluorinated compounds in the solution.

Clause 10. The process of any of clauses 1-9, wherein the first defluorinating species can defluorinate at least one species of fluorinated compound selected from the group consisting of trifluoromethyl arenes, aromatic fluorides, difluoroalkenes, per- and polyfluoroalkyl substances, and sulfur hexafluoride. A technical effect of this can be that the defluorinating species substantially removes the species of fluorinated compound from the mixture or partially defluorinates the species of fluorinated compound.

Clause 11. A system, comprising: a first reactor, wherein the first reactor is configured to: receive a mixture of fluorinated compounds; and partially defluorinate, by a first defluorinating species, the mixture of fluorinated compounds, wherein the first defluorinating species is a solid supported species; and at least one additional reactor, wherein the at least one additional reactor is configured to substantially defluorinate, by at least one additional defluorinating species, the partially defluorinated mixture of fluorinated compounds. A technical effect of this system can be removing fluorinated compounds from a mixture of compounds.

Clause 12. The system of clause 11, wherein the partially defluorinating the mixture of fluorinated compounds comprises partially defluorinating a first species of fluorinated compound in the mixture of fluorinated compounds. This can have the technical effect of enabling subsequent defluorination reactions with the partially defluorinated first species.

Clause 13. The system of clause 12, wherein the substantially defluorinating the mixture of fluorinated compounds comprises completely defluorinating the partially defluorinated first species of fluorinated compound. This can have the technical effect of substantially removing the first species from the mixture of compounds.

Clause 14. The system of any of clauses 11-13, wherein the partially defluorinating the mixture of fluorinated compounds comprises completely defluorinating a first species of fluorinated compound in the mixture of fluorinated compounds. This can have the technical effect of substantially removing the first species from the mixture of compounds.

Clause 15. The system of any of clauses 11-14, wherein the first defluorinating species is a photocatalyst. This can have the technical effect of defluorinating compounds via photocatalyzed reaction.

Clause 16. The system of clause 15, wherein the photocatalyst is selected from the group consisting of [4,4′-bis(1,1-dimethylethyl)-2,2′-bipyridine-N1,N1′]bis[2-(2-pyridinyl-N)phenyl-C]iridium(III) hexafluorophosphate, tris(2,2′-bipyridine)ruthenium(II) hexafluorophosphate, N-phenylphenothiazine, 1,2,3,5-tetrakis(carbazol-9-yl)-4,6-dicyanobenzene, and [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate. This can have the technical effect of enabling photocatalyzed reactions with one or more fluorinated species.

Clause 17. The system of any of clauses 11-14, wherein the first defluorinating species is selected from the group consisting of a palladium hydrogenation catalyst, a photocatalyst, a sequestration polymer, and an alkali metal. The palladium hydrogenation catalyst, photocatalyst, and alkali metal can have the technical effect of defluorinating fluorinated compounds, and the sequestration polymer can have the technical effect of sequestering fluorinated compounds.

Clause 18. The system of clause 17, wherein: the at least one additional reactor is a photoreactor; and the at least one additional defluorinating species comprises hydrated electrons. The technical effect of the photoreactor can be generating the hydrated electrons in an aqueous solution. The hydrated electrons may then hydrogenate fluorinated compounds in the solution.

Clause 19. The system of any of clauses 11-18, wherein the first defluorinating species can defluorinate at least one species of fluorinated compound selected from the group consisting of trifluoromethyl arenes, aromatic fluorides, difluoroalkenes, per- and polyfluoroalkyl substances, and sulfur hexafluoride. A technical effect of this can be that the defluorinating species substantially removes the species of fluorinated compound from the mixture or partially defluorinates the species of fluorinated compound.

Clause 20. A system, comprising: a set of reactors configured to operate in sequence to substantially defluorinate a mixture of fluorinated compounds, wherein the operating in sequence comprises: receiving, at a first reactor from the set of reactors, the mixture of fluorinated compounds; defluorinating, by a first defluorinating species in the first reactor, a first species of fluorinated compound in the mixture of fluorinated compounds; and defluorinating, by a second defluorinating species in a second reactor from the set of reactors, a second species in the mixture of fluorinated compounds. A technical effect of this system can be removing fluorinated compounds from a mixture of compounds.

Clause 21. A process, comprising: substantially defluorinating, by a set of reactors configured to operate in sequence, a mixture of fluorinated compounds, wherein the substantially defluorinating comprises: receiving, at a first reactor from the set of reactors, the mixture of fluorinated compounds; defluorinating, by a first defluorinating species in the first reactor, a first species of fluorinated compound in the mixture of fluorinated compounds; and defluorinating, by a second defluorinating species in a second reactor from the set of reactors, a second species in the mixture of fluorinated compounds. A technical effect of this process can be removing fluorinated compounds from a mixture of compounds.

Clause 22. A system, comprising: a set of reactors configured to operate in sequence to substantially defluorinate a mixture of fluorinated compounds, wherein: the set of reactors comprises a reactor containing a first defluorinating species on a solid support; the first defluorinating species is an alkali metal; and the solid support is an oxide. A technical effect of this system can be removing fluorinated compounds from a mixture of compounds.

Clause 23. The system of clause 22, wherein the reactor also contains a solution of a redox mediator. A technical effect of this can be allowing the alkali metal to react with fluorinated species at lower temperatures than without the redox mediator.

Clause 24. The system of clause 22, wherein the reactor also contains bare silica. This can have the technical effect of preventing alkali metal vapor from leaving the reactor.

Clause 25. The system of any of clauses 22-24, wherein: the set of reactors further comprises a next reactor containing a second defluorinating species on a second solid support; and the second defluorinating species is selected from the group consisting of a hydrogenation catalyst, a photocatalyst, and a sequestration polymer. This can have the technical effect of defluorinating species that were not removed by the first reactor.

Various embodiments of the present disclosure are described herein with reference to the related drawings, where like numbers refer to the same component. Alternative embodiments can be devised without departing from the scope of the present disclosure. It is noted that various connections and positional relationships (e.g., over, below, adjacent, etc.) are set forth between elements in the following description and in the drawings. These connections and/or positional relationships, unless specified otherwise, can be direct or indirect, and the present disclosure is not intended to be limiting in this respect. Accordingly, a coupling of entities can refer to either a direct or an indirect coupling, and a positional relationship between entities can be a direct or indirect positional relationship. As an example of an indirect positional relationship, references in the present description to forming layer “A” over layer “B” include situations in which one or more intermediate layers (e.g., layer “C”) is between layer “A” and layer “B” as long as the relevant characteristics and functionalities of layer “A” and layer “B” are not substantially changed by the intermediate layer(s).

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

For purposes of the description hereinafter, the terms “upper,” “lower,” “right,” “left,” “vertical,” “horizontal,” “top,” “bottom,” and derivatives thereof shall relate to the described structures and methods, as oriented in the drawing figures. The terms “overlying,” “atop,” “on top,” “over,” “positioned on,” or “positioned atop” mean that a first element, such as a first structure, is present on a second element, such as a second structure, wherein intervening elements such as an interface structure can be present between the first element and the second element. The term “direct contact” means that a first element, such as a first structure, and a second element, such as a second structure, are connected without any intermediary conducting, insulating or semiconductor layers at the interface of the two elements. It should be noted, the term “selective to,” such as, for example, “a first element selective to a second element,” means that a first element can be etched, and the second element can act as an etch stop.

As used herein, terms such as “standard conditions,” “ambient conditions,” “room temperature,” etc. are given their ordinary meanings, as will be understood by persons of ordinary skill in the art. For example, the term “standard conditions” can refer to any appropriate temperature and pressure within a range corresponding to standard temperature and pressure (STP), normal conditions (NC), or standard laboratory conditions (SLC) set by standards organizations such as the International Union of Pure and Applied Chemistry (IUPAC) or National Institute of Standards and Technology (NIST), e.g., temperatures ranging from about 0° C. to 28° C. and a pressure of about 100 kPa. In another example, the term “room temperature” can refer to temperatures between approximately 20° C. and 30° C. (e.g., ~25-28° C). Further, the term “ambient conditions” refers to the actual temperature and pressure at the location and time in which a process is being carried out. The temperature and pressure variations that can be encompassed by “standard conditions,” “ambient conditions,” and “room temperature” can depend on the type of reaction and/or other factors known to persons of ordinary skill in the art.

As used herein, the articles “a” and “an” preceding an element or component are intended to be nonrestrictive regarding the number of instances (i.e., occurrences) of the element or component. Therefore, “a” or “an” should be read to include one or at least one, and the singular word form of the element or component also includes the plural unless the number is obviously meant to be singular.

As used herein, the terms “invention” or “present invention” are non-limiting terms and not intended to refer to any single aspect of the particular invention but encompass all possible aspects as described in the specification and the claims.

Unless otherwise noted, ranges (e.g., time, concentration, temperature, etc.) indicated herein include both endpoints and all numbers between the endpoints. Unless specified otherwise, the use of a tilde (~) or terms such as “about,” “substantially,” “approximately,” “slightly less than,” and variations thereof are intended to include the degree of error associated with measurement of the particular quantity based upon the equipment available at the time of filing the application. For example, “about” can include a range of ±8% or 5%, or 2% of a given value, range of values, or endpoints of one or more ranges of values. Unless otherwise indicated, the use of terms such as these in connection with a range applies to both ends of the range (e.g., “approximately 1 g-5 g” should be interpreted as “approximately 1 g-approximately 5 g”) and, in connection with a list of ranges, applies to each range in the list (e.g., “about 1 g-5 g, 5 g-10 g, etc.” should be interpreted as “about 1 g-about 5 g, about 5 g-about 10 g, etc.”).

As described herein, compounds of the present disclosure can optionally be substituted with one or more substituents, as exemplified by particular classes, subclasses, and species of the present disclosure. As described herein, any of the above moieties or those introduced below can be optionally substituted with one or more substituents described herein. However, it is generally known that the substituents should be selected so that they do not adversely affect the useful properties of the compound or its function. The term “substituted” in the context of the present disclosure means that one or more hydrogen atoms of the indicated radical or group is/are independently replaced by the same or a different substituent(s). Additionally, the term “substituted” specifically provides for one or more, e.g., two, three, or more, substituents commonly used in the art.

As used herein the term “aliphatic” encompasses the terms alkyl, alkenyl, or alkynyl. Aliphatic radicals or groups may have any degree of saturation, such as groups having only single carbon-carbon bonds (“alkyl” or “alkylene”), groups having one or more double carbon-carbon bonds (“alkenyl”), radicals having one or more triple carbon-carbon bonds (“alkynyl”), and groups having a mixture of single, double and/or triple carbon-carbon bonds.

As used herein, an “alkyl” group refers to a saturated aliphatic hydrocarbon group containing at least one carbon atom (e.g., C1-C4, C1-C6, or C1-C8 alkyls). An alkyl group can be straight, branched, cyclic, or any combination thereof. Unless specifically limited otherwise, the term “alkyl,” as well as derivative terms such as “alkoxy” and “thioalkyl,” as used herein, include within their scope, straight chain, branched chain, and cyclic moieties. If the alkyl radical is further bonded to another atom, it becomes an alkylene radical or alkylene group. In other words, the term “alkylene” also refers to a divalent linear or branched alkyl. For example, —CH₂CH₃ is an ethyl, while —CH₂CH₂— is an ethylene. The term “alkylene” alone or as part of another substituent refers to a saturated linear or branched divalent hydrocarbon radical obtained by removing two hydrogen atoms from a single carbon atom or two different carbon atoms of a starting alkane.

Examples of alkyl radicals/moieties or alkyl groups include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl. The alkyl group or alkylene group as defined above may be unsubstituted or substituted with one or more substituents as set forth below.

As used herein, the term “cyclic” refers to a ring compound or group comprising at least three carbon atoms and the bonds between pairs of adjacent atoms may all be of the type designated single bonds (involving two electrons), or some of them may be double or triple bonds (with four or six electrons, respectively). Examples of cyclic aliphatic groups can include phenyl, saturated cycloalkyls (e.g., cyclopropyl, cyclobutyl, cyclopentyl, or cyclohexyl), etc.

Monocyclic aromatic hydrocarbon groups are composed of a single aromatic ring, for example benzene, toluene, ethylbenzene, and xylenes. Bicyclic aromatic hydrocarbon groups can contain two benzene rings, such as naphthalene. The aromatic hydrocarbons groups can possess one or more aromatic rings in their structures. Aromatic hydrocarbons are unsaturated hydrocarbons with sigma bonds and pi-electrons that are delocalized between carbon atoms forming a circle. In contrast, aliphatic hydrocarbons lack this delocalization. The aromatic ring according to the present disclosure can be a four-, five-, six-, or seven-membered ring. In some embodiments, the aromatic hydrocarbons have the general chemical formula CₙHₙ. In some embodiments, the aromatic hydrocarbon group contains a benzene ring.

Modifications or derivatives of the compounds disclosed throughout this specification are contemplated as being useful with the methods and compositions of the present disclosure. Derivatives may be prepared and the properties of such derivatives may be assayed for their desired properties by any method known to those of skill in the art. In certain aspects, “derivative” refers to a chemically modified compound that still retains the desired effects of the compound prior to the chemical modification.

Herein, “particle” refers to solid nanoparticles or microparticles having an approximately spherical shape. The term “particle size” refers to a particle size evaluated for a spherical object. However, the shapes of particles may be irregular and non-spherical. Therefore, particle size dimensions are based on replacing a given particle with an imaginary sphere having properties (e.g., volume, weight, area, and/or drag coefficient) of the particle. In other embodiments, silica particles can have other structures, such as rods.

The size of the sphere can be expressed in various ways (e.g., volume, diameter, radius, weight, surface area, etc.). Herein, particle size is defined by diameter unless otherwise specified. For example, “a 200 nm particle” would refer to a particle having a diameter of 200 nm. In real systems, particles are generally present as ensembles having different sizes, and the size for a given ensemble can be described by an average particle size (e.g., median size, geometric mean size, or average size) or a particle size distribution, which is a mathematical function or a list of values that defines average particle size obtained for a sample of particles, sorted according to size.

Turning now to an overview of technologies that are more specifically relevant to aspects of the present disclosure, in general, in semiconductor manufacturing, fluorinated and other process gases provide selectivity and specificity in processes such as etching, deposition, and chamber cleaning. Fluorinated compounds (e.g., tetrafluoromethane (CF₄), trifluoromethane (CHF₃), sulfur hexafluoride (SF₆,) etc.) are used in a wide variety of industrial applications and consumer products. For example, compounds such as CF₄ are used to pattern semiconductor wafers and multiple materials layers thereon, predominantly containing silicon or silicon compounds. The resulting exhaust contains a small fraction of silicon tetrafluoride (SiF₄) and a larger fraction of unreacted or partially reacted fluorinated compounds.

Gases containing fluorinated compounds are major components of direct (scope 1) and indirect (scope 2) greenhouse emissions for semiconductor fabrication plants (“fabs”). Some of these fluorinated compounds (e.g., NF₃) are reactive and can be reliably scrubbed from the exhaust of fabrication tools. However, many of the fluorinated compounds used in significant quantities (e.g., per- and polyfluoroalkyl substances (PFAS), SF₆, CF₄, CHF₃, etc.) are relatively inert and therefore difficult to capture or recover. Scrubbing systems for fluorocarbons and other exhaust gases are currently mandatory in most industrial applications, with wet scrubbing being a common practice. There are also challenges in breaking down larger fluorinated compounds, such as PFAS, for disposal or recycling.

Specific hurdles to abatement of gaseous fluorocarbons, PFAS, etc. include their high fugacity, high stability, low polarity, and low solubility in water. Additionally, the reuse of captured fluorinated gases in semiconductor manufacturing can be hindered by the extreme purity requirements of this industry. For example, the evolution of contaminants during wafer processing and the low volumes of gases recovered using existing techniques can limit the effectiveness of on-site purification and reuse installations.

Consequently, destructive approaches are commonly employed to eliminate fluorinated gases and other exhaust gases (e.g., chlorinated gases). Given the toxicity and flammability of byproducts in the semiconductor exhaust gas mixtures, the byproducts are often subjected to incineration followed by scrubbing of soluble species. While chlorinated compounds can be converted in this process, the high stability of carbon-fluorine (C—F) bonds makes it difficult to break down fluorinated compounds by this approach. CF₄ is considered to be particularly resilient to incineration, requiring temperatures in excess of 1400° C. This resilience to abatement is due to the fact that C—F bonds are the strongest in organic chemistry and are further strengthened by each additional fluorine atom, 4 being the maximum. Consequently, attempts to abate larger PFAS molecules by industry-standard incineration methods in most cases leads to the formation of CF₄ as a biproduct. Fluorocarbon destruction using plasma and/or catalyst-assisted approaches have been proposed, but require significant investment in equipment and energy, further increasing the carbon footprint of the fab.

Other approaches can include separation of fluorinated species from exhaust gas followed by further refinement on a larger scale, e.g., at a waste treatment facility. Separation techniques can include cryogenic liquefaction and distillation of fluorinated constituents, continuous chromatography, membrane separation, and solid sorbents. Other separation techniques can include absorption into a carrier liquid or into a porous carbon or metal organic framework. A disadvantage of these absorption strategies can be that fluorination imparts solution properties significantly different from typical hydrocarbons. To facilitate absorption of fluorocarbons, the absorption medium itself may have to be chemically fluorinated to enhance the interaction energies between the target gases and the medium.

Embodiments of the present disclosure may overcome these and other disadvantages of current techniques for fluorinated compound abatement. In some embodiments, a variety of fluorinated compounds may be destroyed or captured in an defluorination system that includes one or more reactors containing defluorinating species (e.g., polymers that can sequester fluorinated compounds, catalysts or other reactive species that can defluorinate molecules, etc.) on solid support materials. Herein, reactive species and sequestration polymers on solid support materials are referred to as “supported defluorinating species” or “supported species”. The supported species may be covalently bound to a solid support material, absorbed by the solid support material, adsorbed onto a solid support material, or otherwise attached or incorporated into to the solid support material such that the species is not free (e.g., dissolved or suspended) in a liquid or gas phase reaction mixture.

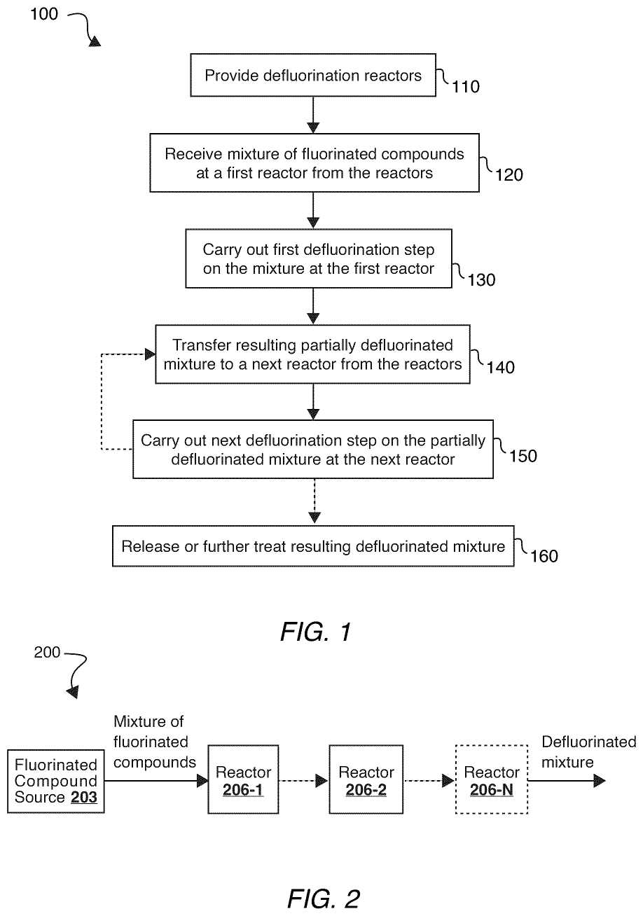

Herein, unless specified otherwise or clear based on context, “defluorination” of a mixture of fluorinated compounds can refer to partial or complete defluorination of the mixture (e.g., by chemical reactions with and/or sequestration of fluorinated molecules). Herein, “complete” defluorination of a mixture of fluorinated compounds refers to defluorination resulting in a substantially defluorinated mixture of compounds, such as a mixture with a concentration (e.g., in micrograms (μg) per liter (L), parts-per-million (ppm), etc.) of fluorinated species below a threshold limit, which may be a recommended or required threshold set by a governing organization, a detection limit, etc. For example, a substantially defluorinated liquid mixture may have a fluorinated compound concentration less than a threshold between about 5×10⁻⁶ μg/L and 1 μg/L. Herein, “partial” defluorination of a mixture of fluorinated compounds refers to defluorination resulting in a mixture of compounds with a concentration of fluorinated compounds higher than the threshold limit. This is discussed in greater detail below with respect to FIG. 1. The defluorination environment may advantageously allow mixtures of fluorocarbons with various properties to be destroyed via one or more catalytic reactions in a single system. For example, the use of solid supports may allow a greater number of catalysts/reaction environments to be used than in existing techniques because, e.g., the solid supports can allow safer handling of highly reactive materials (e.g., alkali metals), better access to catalysts that are otherwise difficult to use in large scale/industrial settings (e.g., organometallic or organic photocatalysts), etc. Further, the defluorination environment may convert fluorinated compounds into smaller species readily scrubbed by water or alkaline solutions.

In some embodiments, the defluorination system includes a reactor with metal, organic, or organometallic catalysts on silica, alumina, or other solid support. The catalysts can defluorinate various types of fluorinated compounds via reduction reactions. In some embodiments, a liquid- or gas-phase redox mediator may facilitate these reactions. In further embodiments, two or more supported defluorinating species may be used simultaneously or in sequence in order to destroy a mixture of fluorinated compounds, which may have different reactivities and physical properties. For example, a first reactor may contain species capable of partially or fully defluorinating certain species in a mixture of fluorinated compounds. In a next reactor, partially defluorinated compounds may be fully defluorinated and/or unreacted fluorinated species in the mixture may be defluorinated.

Referring now to the drawings, in which like numerals represent the same or similar elements, FIG. 1 is a flow diagram illustrating a process 100 of defluorination, according to some embodiments. Process 100 is described herein with reference to FIG. 2, which is a block diagram illustrating a system 200 for defluorination (e.g., via process 100), according to some embodiments.

Reactors 206-1, 206-2, 206-N (collectively referred to herein as “reactors 206”) for defluorination can be provided. This is illustrated at operation 110. While system 200 is illustrated with multiple reactors 206, in other embodiments, system 200 may include only one reactor 206-1. Additionally, system 200 may include additional components that are not illustrated in FIG. 2. Examples of these compounds are discussed in greater detail below.

At least one of reactors 206 may contain a supported defluorinating species (e.g., catalyst or sequestration polymer) and, as appropriate, additional reagents for defluorination of one or more fluorinated species. In some embodiments, the solid support for the reactive species is an oxide, such as silica (SiO₂) or alumina (Al₂O₃)), although any appropriate solid support may be used, e.g., polymer-based (e.g., polystyrene beads), other oxides (e.g., titania (TiO₂), ceria (CeO₂), iron oxide (e.g., Fe₃O₄), or magnesium oxide (MgO)), metals, etc. Depending on the types of defluorinating species and solid support, the defluorinating species may be incorporated into the solid support via absorption or attached to the solid support surface, e.g., via covalent binding or adsorption.

The solid support can be in any appropriate form with sufficient surface area to allow contact/reactions between the supported reactant and molecules of the fluorinated compounds from source 203. For example, the supported fluorinating species may be supported by particles with average diameters ranging from 1 nm to 1 mm. The solid support material may optionally be porous (e.g., amorphous silica with an average pore size of about 15 nm) or nonporous. In some embodiments, the solid support material can be in the form of granules, nanoparticles, microparticles, powder, pellets, fiber (e.g., a wool or sponge), shavings, etc.

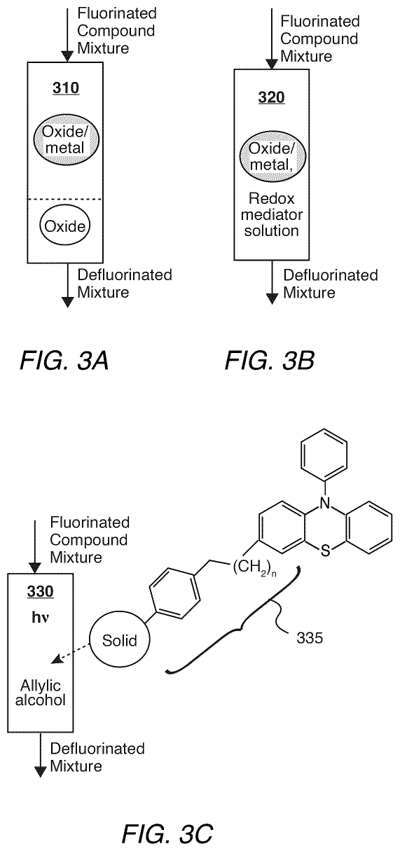

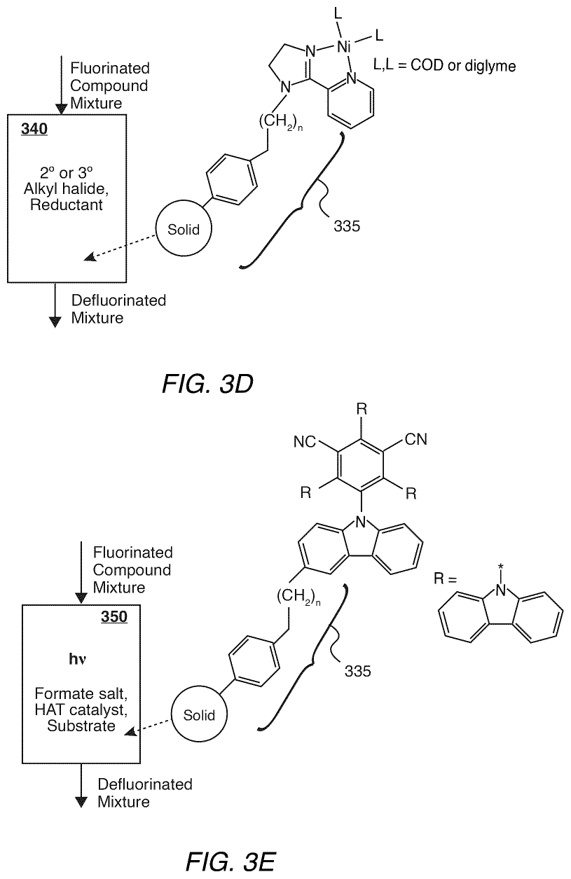

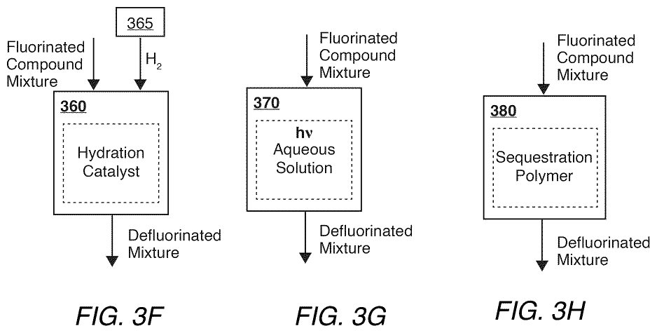

In some embodiments, reactors 206 include at least one photoreactor. For example, a photoreactor from reactors 206 may include a supported photocatalyst (see, e.g., FIG. 3E). In some embodiments, one or more of reactors 206 may have a defluorinating species that is not on a solid support (e.g., hydrated electrons). An example of this is discussed in greater detail with respect to FIG. 3G. Reactors 206 may also include a sequestration component that, rather than carrying out defluorination reactions, sequesters at least a portion of the fluorinated compounds. Herein, “sequestration” refers to techniques for removing fluorinated compounds from a gas or solution by capturing/trapping the fluorinated compounds. Illustrative examples of various defluorination reactors 206 are discussed in greater detail below with respect to FIGS. 3A-3H.

Reactors 206 may be selected from, e.g., reactors 310-380 illustrated in FIGS. 3A-3H. In some embodiments, reactors 206 can include at least one packed bed reactor or any heterogeneous reactor (e.g., catalytic reactor) appropriate for using a catalyst or other defluorinating species on a solid support (“supported defluorinating species”) to defluorinate one or more species of fluorinated compound in process 100. For example, reactors 206 may include a fixed bed reactor, trickle bed reactor, moving bed reactor, rotating bed reactor, fluidized bed reactor, slurry bed reactor, etc. Depending on the supported defluorinating species, one or more additional reagents can be present in reactors 206 as well (e.g., a redox mediator, consumable reagent, reaction substrate, etc.). This is discussed in greater detail below.

When organic or organometallic defluorinating species are used in reactors 206, these compounds may have reactive functional groups that form covalent bonds with surface functionalities on the solid support. For example, a defluorinating species (e.g., catalyst) modified to include an organic sidechain having hydroxyl groups may bind to silica or other oxide surfaces. However, any appropriate techniques for surface functionalization of solid support materials known in the art may be used to attach defluorinating species to a solid support (e.g., by chemical bonds such as covalent, ionic, and/or hydrogen bonds). In some embodiments, a defluorinating species-functionalized monomer may be polymerized or copolymerized with another monomer to form defluorinating species-functionalized polymer particles (e.g., polystyrene particles). For example, styrene monomers may be functionalized to include a catalyst as those shown in FIGS. 3C-3E.

A first reactor 206-1 from reactors 206 can receive fluorinated compounds from a fluorinated compound source 203 (“source 203”). This is illustrated at operation 120. For example, reactor 206-1 can receive a mixture of fluorinated species from source 203. Reactor 206-1 may receive the fluorinated compounds in a carrier liquid (e.g., an ethereal solvent) or carrier gas. In some embodiments, mixtures of compounds from the fluorinated compound source 203 may be processed or treated by additional components of system 200 (not shown) before entering reactor 206-1. For example, one or more species (e.g., non-fluorinated waste compounds, solids, etc.) may be destroyed, scrubbed, or otherwise removed from the received mixture prior to operation 120.

In some embodiments, source 203 may be a semiconductor fab. For example, a wafer lot from a semiconductor fab can be provided for production of integrated circuits by wafer processing equipment, such as tools for plasma etching. Plasma etching can use volatile PFAS (e.g., CF₄, CHF₃, etc.) and other fluorinated compounds (e.g., NF₃) to pattern semiconductor wafers and other material layers thereon. Therefore, the fluorinated compounds received at operation 120 may be fluorinated gases in an exhaust gas from semiconductor processing. The fluorinated compounds received by reactor 206-1 may be mixed with other exhaust and/or carrier gases.

In further embodiments, the fluorinated compounds received by reactor 206-1 may include byproducts of industrial synthesis reactions. For example, source 203 may be a facility that produces polymers such as polytetrafluoroethylene (PTFE). In additional embodiments, source 203 may be a facility for collecting and disposing of PFAS waste.

In some embodiments, the fluorinated compounds received at operation 120 may include, but are not limited to, fluorinated species such as CF₄, CHF₃, SF₆, perfluoroheptanoic acid and/or other perfluorocarboxylic acids (PFCAs (CₙF₂ₙ₊₁COOH, where n is a positive integer (e.g., 7, 8, or 9)), trifluoroacetate (TFA), fluorotelomer carboxylic acids (FTCAs (CₙF₂ₙ₊₁—CH₂CH₂—COOH, where n is a positive integer)), perfluoropentanesulfonic acid and or other perfluoroalkanesulfonic acids (CₙF₂ₙ₊₁SO₃⁻, where n is a positive integer (e.g., 4, 6, or 8), per- and polyfluoro dicarboxylic acids (PFdiCAs (HOOC—CₙF₂ₙ—COOH)), 2-perfluoropropylethanol, difluoroalkenes, trifluoromethyl arenes, etc.

The received fluorinated mixture can be at least partially defluorinated via reactions with or sequestration of fluorinated species in the first reactor 206-1. This is illustrated at operation 130. For example, the first reactor 206-1 may partially defluorinate the mixture of fluorinated compounds from source 203 in a first defluorination step using a reactor selected from reactors 310-380 (see below). While not shown in FIG. 1, depending on the mixture of compounds received at operation 120 and the reaction environment in the first reactor 206-1, the mixture of compounds from source 203 may be completely defluorinated at operation 120 in some embodiments.

The partially defluorinated mixture can then be transferred to a next reactor 206-2 from reactor 206-1. This is illustrated at operation 140. The partially defluorinated mixture can be a gas- or liquid-phase mixture (e.g., including a carrier gas or solvent, defluorination reaction products, unreacted species, etc.). The fluorinated compounds in the partially defluorinated mixture may include partially defluorinated compounds, unreacted fluorinated compounds, or both. In some embodiments, the concentration of fluorinated compounds is lower than in the starting mixture because of destruction and/or sequestration of fluorinated compounds in the mixture. A partially defluorinated mixture of compounds may also refer to a mixture in which molecules of one or more fluorinated species in the mixture have been partially defluorinated (e.g., where SF₆ in the mixture is converted to SF₅). In further embodiments, in a partially defluorinated mixture, molecules of some but not all fluorinated species in the mixture have been fully defluorinated (e.g., where SF₆ in the mixture is converted to sulfur(S) while CF₄ is substantially unreacted) and/or partially defluorinated (e.g., where CF₄ in the mixture is converted to carbon (C), larger perfluorocarbons are partially defluorohydrogenated, and SF₆ is unreacted).

Fluorinated compounds in the partially defluorinated mixture from the first reactor 206-1 can be at least partially defluorinated or sequestered in a second defluorination step in the second reactor 206-2. This is illustrated at operation 150. While not shown in FIG. 1, two or more of reactors 206 may operate simultaneously at operation 130 and/or 150 in some embodiments. The reaction environment in reactor 206-1 may include a different solid supported defluorinating species and, in some embodiments, other reactive species than the reaction environment in reactor 206-2. Examples of reactive species that can be included in reactors 206 are discussed in greater detail below with respect to FIGS. 3A-3H. As illustrated by the dashed arrows in FIG. 1, after the next defluorination of the partially defluorinated mixture in reactor 206-2 (operation 150), the resulting at least partially defluorinated mixture may be transferred to a next reactor 206-N to be further defluorinated (repeating operations 140 and 150), transferred to another component (not shown) of system 200, or released (see below at operation 160). The transfer and treatment in a next reactor may optionally be repeated with at least one additional reactor from reactor(s) 206-N. That is, operations 140 and 150 may be repeated with one or more reactors selected, e.g., from the reactors illustrated in FIGS. 3A-3H in some embodiments.

A defluorinated mixture of compounds containing unreacted species and/or products of reactions in reactors 206-1, 206-2, and optional next reactor(s) 206-N may be released or transferred to another component of system 200 (not shown), such as a separation component, to be treated further. This is illustrated at operation 160. In some embodiments, the defluorinated mixture of compounds at operation 160 is completely defluorinated. The defluorinated mixture can be a gas- or liquid-phase mixture (e.g., including a carrier gas or solvent, defluorination reaction products, unreacted compounds, etc.). Scrubbing methods known in the art may be used to treat the defluorinated mixture at operation 160. For example, wet scrubbing, gas dilution, or other techniques known in the art may be used. In some embodiments, a gaseous defluorinated mixture may travel from reactor 206-N into a lower-temperature component (e.g., a cooling and/or separation component) that allows condensation of products such as carbon and fluorides.

In some embodiments, the defluorinated mixture is transferred to a separation component (not shown) of system 200 at operation 160. The separation component can include one or more chambers, filters (e.g., a carbon filter), reagents, etc. For example, solvents and defluorination products in the defluorinated mixture, such as fluorides (e.g., NaF, KF, etc.), carbon, sulfur, etc. can be condensed in a cooling chamber (not shown). In some embodiments, the separation component uses a coolant to facilitate condensation in a cooling chamber, reaction chamber, filtration component, etc.

Examples of separation components may include a filter, cyclone, chemical absorbent, physical absorbent, and/or other component for isolating, reacting, and/or purifying the defluorinated mixture. For example, a cyclone and/or a sleeve filter may be used to aid separation of condensed materials from the defluorinated gas. The separation component may be integrated with a commercially available stainless steel bag filter housing. In some embodiments, additional reagents in solid, gaseous, or liquid phase, as well as chemical (e.g., NaHCO₃ or NaF) and/or physical absorbents can be used to facilitate efficient PFAS abatement and/or separation of the reaction products to obtain defluorinated exhaust gas.

System 200 may also include components for monitoring defluorination (not shown) at one or more steps in process 100. This may enable automation of one or more steps in process 100. Examples of monitoring components that may be used can include inline flow meters, Fourier-transform infrared (FTIR) spectroscopy modules, mass spectrometers, temperature sensors, digital cameras, microscopes, optical sensors, and/or electrical current sensors.

FIGS. 3A-3H are schematic diagrams illustrating example reactors 310-380 with supported defluorinating species, according to some embodiments. Examples of fluorinated species in a mixture of fluorinated compounds that may react with the supported defluorinating species are discussed below with respect to FIGS. 3A-3H. However, the supported defluorinating species in reactors 310-380 may react with/defluorinate other fluorinated species present in the mixture as well. Further, as will be understood by persons of ordinary skill, there may be fluorinated compounds in the mixture that do not react with (or are not sequestered by) the supported defluorinating species.

Reactors 310-380 can be examples of reactors 206 from system 200 used in process 100. In each of FIGS. 3A-3H, an arrow entering a reactor 310-380 represents the transfer of fluorinated compounds from source 203 (e.g., at operation 120) or from a previous reactor 206-1, 206-2, or 206-N (e.g., at operation 140), and an arrow exiting a reactor 310-380 represents the transfer of a defluorinated mixture (e.g., at operation 140 or 160) resulting from treatment at operation 130 or 150, respectively. This defluorinated mixture may be substantially defluorinated (e.g., at operation 160) or partially defluorinated (e.g., at operation 140).

In some embodiments, the defluorinated mixture exiting a reactor from reactors 310-380 contains fluorinated compounds that are transferred to a next reactor (e.g., at operation 140). In some embodiments, reactors 206 include more than one of reactors 310-380 and/or other reactors that are not illustrated in FIGS. 3A-3H (e.g., other reactors discussed herein and/or defluorination or other dehalogenation reactors known to those of ordinary skill in the art).

In some embodiments, reactors 206 include a reactor 310 (FIGS. 3A) and/or 320 (FIG. 3B) where the solid supported defluorinating species is an alkali metal absorbed into an oxide solid support, such as silica or alumina. In some embodiments, the alkali metal is sodium (Na), potassium (K), or an alloy thereof (e.g., NaK). In some embodiments, absorption of the alkali metal is accomplished by bringing the alkali metal in its liquid state into contact with the oxide. For example, the supported defluorinating species in reactors 310 and 320 may be manufactured at room temperature using NaK (wt. % 20/80) and silica as the solid support. In some embodiments, the absorbed alkali metal in reactors 310 and 320 may be safely handled at room temperature and low humidity because of its absorption into the oxide solid support.

During operation 120 or 140, reactor 310 can receive fluorinated compounds at a temperature sufficient for vaporizing the alkali metal (e.g., about 400-600° C). When the fluorinated compounds enter reactor 310, volatile fluorinated compounds (e.g., CF₄) may be reduced in a vapor phase reaction with the alkali metal, resulting in carbon and a metal fluoride, such as NaF and/or KF. In some embodiments, the fluorinated gas may also react with silica supporting the alkali metal. In addition to the alkali metal supported species, reactor 310 may include at least one additional reagent. For example, as shown in FIG. 3A, in order to prevent alkali metal vapor from exiting the reactor with the carrier gas, reactor 310 can include a cooler region containing bare silica (silica substantially without absorbed alkali metal) to absorb unreacted alkali metal. In other embodiments, the cooler region/bare silica may be in a subsequent component of system 200, such as a separation component (not shown). The gas flow may also be maintained using a carrier gas (e.g., N₂) and backpressure regulators.

In further embodiments, during operation 120 or 140, reactor 320 can receive a mixture of fluorinated compounds that include compounds that normally require higher temperatures than CF₄, such as SF₆. Reactor 320 may defluorinate these compounds (operation 130 or 150) at lower temperatures than with the alkali metal alone as in reactor 310 by using a solution of a redox mediator, such as a fused polycyclic aromatic compound. For example, SF₆ may be defluorinated in reactor 320 at ambient temperatures using naphthalene as a redox mediator in an organic solvent.

In some embodiments, reactors 206 may include a reactor 330 (FIG. 3C) that can defluorinate SF₆ under standard or ambient conditions via a solid supported photocatalyst in the presence of light (hν). In some embodiments, reactor 330 is a photoreactor. FIG. 3C illustrates a chemical structure diagram of such a supported species in reactor 330 where the photocatalyst is N-phenylphenothiazine bound to a polystyrene solid support via a linking group 335 (e.g., where n=4). In other embodiments (not shown), the supported defluorinating species in reactor 330 may be an iridium photocatalyst such as [4,4′-bis(1,1-dimethylethyl)-2,2′-bipyridine-N1,N1′]bis[2-(2-pyridinyl-N)phenyl-C]iridium(III) hexafluorophosphate (Ir(ppy)₂(dtbbpy)PF₆), or a ruthenium photocatalyst, such as tris(2,2′-bipyridine)ruthenium(II) hexafluorophosphate (Ru(bpy)₃(PF₆)₂), on a solid support. In these embodiments, reactor 330 can also contain additional reagents including a stoichiometric reductant, such as diisopropylethylamine (DIPEA), and an allylic alcohol, such as 5-phenylpent-2-en-1-ol (e.g., in an acetonitrile (MeCN) or dichloroethane (DCE) solution).

The reaction environment in reactor 330 can also include a consumable reagent selected from a variety of allylic alcohols. The supported defluorinating species in reactor 330 (e.g., the illustrated photocatalyst or a ruthenium or iridium catalyst such as those discussed above), may catalyze a pentafluorosulfanylation reaction with the allylic alcohol and SF₆ at operation 130 or 150 from a mixture of fluorinated compounds received at operation 120 or 140, respectively.

The allylic alcohol may be a primary (1°) or secondary (2°) allylic alcohol in some embodiments. An iridium or ruthenium photocatalyst and stoichiometric reductant in reactor 330 can carry out photocatalyzed deoxyfluorination of the 1° or 2° allylic alcohol using SF₆ from source 203, resulting in corresponding 1° or 2° allylic fluorides and defluorinated species (e.g., SF₅). For example, upon exposure to blue light in reactor 330, solid supported Ru(bpy)₃(PF₆)₂ can catalyze a reaction with SF₆ received at operation 120 to convert (2E)-5-phenylpent-2-en-1-ol (an allylic alcohol) to [(3E)-5-fluoro-3-penten-1-yl]benzene (the corresponding allylic fluoride).

In some embodiments, linking group 335 is bound to the solid support via, e.g., forming polystyrene beads with a photocatalyst-functionalized co-monomer (e.g., as shown in FIG. 3C). In other embodiments, the photocatalyst may be modified to include a linking group with siloxane functional groups (not shown), which can be bound to a silica solid support. In further embodiments, the photocatalysts shown in reactors 330 and 350 can be bound or adsorbed on the surface of the solid support using other surface functionalization techniques known in the art.

Reactors 206 may include a reactor 340 (FIG. 3D) that can defluorinate aromatic or aliphatic fluorides. In some embodiments reactor 340 may defluorinate difluoroalkenes. The supported defluorinating species in reactor 340 can be a nickel-based organometallic catalyst bound via a linking group such as linking group 335 (e.g., where n=2) to a solid support. In some embodiments, an analogous nickel-based organometallic catalyst with a bidentate ligand other than the illustrated 2-(2′-pyridyl)imidazole, such as a bipyridine- or phenanthroline-family ligand, may be used as a supported defluorinating species. Additional species in reactor 340 participating in defluorination reactions can include an alkyl halide and a reductant. In some embodiments, the alkyl halide is a 2° alkyl halide or a tertiary (3°) alkyl halide. The reductant may be, e.g., Zn, Mn, bis(pinacolato)diboron/K₃PO₄, or diethoxymethylsilane/Na₂CO₃. In some embodiments, at operation 120 or 140, the mixture of fluorinated compounds can include a difluoroalkene. In these embodiments, treatment at reactor 340 (at operation 130 or 150) can result in defluorinative reductive cross-coupling of the difluoroalkene with the alkyl halide. For example, reactor 340 can contain a 3° alkyl halide such as tert-butyl iodide. In this example, if a mixture of fluorinated compounds containing (2,2-difluorovinyl)benzene (a difluoroalkene) is received by reactor 340 (e.g., at operation 120), the resulting defluorinative reductive cross-coupling can result in formation of a partially defluorinated product, [(1Z)-2-fluoro-3,3-dimethyl-1-buten-1-yl]benzene (e.g., at operation 130). The resulting defluorinated mixture containing the partially defluorinated product may be transferred to a next reactor 206-2 or 206-N (e.g., at operation 140) and further defluorinated (e.g., at operation 150).

Reactors 206 may include a photoreactor such as reactor 350 (FIG. 3E) for defluorinating, e.g., trifluoromethyl arenes received at operation 120 or 140. For example, the defluorination reaction in reactor 350 can be a radical defluoroalkylation of the trifluoromethyl arenes. The supported species in reactor 350 can be a photocatalyst bound to an solid support, such as 1,2,3,5-tetrakis(carbazol-9-yl)-4,6-dicyanobenzene (4CzIPN) modified to include linking group 335 (e.g., where n=3) as a sidechain for binding to the solid support, as is illustrated in FIG. 3E. Reactor 350 may contain additional reagents to facilitate the defluoroalkylation, such as a formate salt (e.g., sodium formate), a hydrogen atom transfer (HAT) catalyst (e.g., a thiol, a disulfide, a persulfate, sodium 2-mercaptoethanesulfonate, 1,4-diazabicyclo[2.2.2]octane, etc.), and a substrate (e.g., an aryl ammonium, an aliphatic aldehyde, a sulfonamide, etc.). The radical defluoroalkylation in reactor 350 may partially defluorinate the trifluoromethyl arenes, resulting in difluoroalkyl products.

Although not illustrated herein, other photocatalysts attached to solid supports in a manner substantially similar to that of the photocatalyst in FIG. 3E may be used in at least one of reactors 206 in some embodiments. For example, a photocatalyst such as [Ir(ppy)₂(dtbbpy)]PF₆ or [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate ([Ir{dF(CF₃)ppy}₂(dtbpy)]PF₆) may be similarly bound to the solid support.

In some embodiments, reactors 206 can include a reactor 360 (FIG. 3F) for catalytic hydrogenation of fluorinated compounds from source 203. In some embodiments, the fluorinated compounds include 2-perfluoropropylethanol, perfluoropentanesulfonic acid, and perfluoroheptanoic acid may be received by reactor 360 at operation 120 or 140. For example, reactor 360 can carry out catalytic hydrogenation using hydrogen gas (H₂) and a metal catalyst, such as palladium (e.g., palladium nanoparticles), or an organometallic catalyst such as heterodimetallic complex of ruthenium and palladium. The hydrogen gas can be supplied to reactor 360 from any appropriate hydrogen gas source 365 used in conventional catalytic hydrogenation reactors. In some embodiments, the metal or organometallic hydrogenation catalyst (defluorinating species) can be deposited on a non-porous hollow fiber membrane (solid support) to allow delivery of the hydrogen gas directly to the metal catalyst in a bubble-free manner. This can overcome challenges associated with hydrogen's low solubility in aqueous solutions carrying the fluorinated species.

In some embodiments, reactors 206 can include a photoreactor such as reactor 370 (FIG. 3G) that can carry out catalytic hydrogenation of fluorinated species such as PFCAs, TFA, FTCAs, perfluoroalkanesulfonic acids, PFdiCAs, etc. in an aqueous solution via hydrated electrons generated by UV light (e.g., about 254 nm) reacting with H₂O. For example, a mixture containing fluorinated compounds such as 2-perfluoropropylethanol, perfluoropentanesulfonic acid, and perfluoroheptanoic acid may be received by reactor 370 at operation 120 or 130. In some embodiments, the aqueous solution also includes Na₂SO₃ and NaHCO₃ at about pH 9.5 and 20° C.

In some embodiments, reactors 206 include at least one reactor 380 (FIG. 3H) containing a supported species that can sequester fluorinated compounds such as PFCAs, TFA, FTCAs, perfluoroalkanesulfonic acids, PFdiCAs, etc. For example, perfluorinated compounds may be sequestered by a polymer (“sequestration polymer”) on a solid support. In other examples, a sequestration polymer may be used without solid support. In some embodiments, the supported sequestration polymer may be a polymer with carbonyl moieties, such as a polyimide, polyester, polymethylmethacrylate (PMMA), polyetherimide (e.g., poly(bisphenol A-co-4-nitrophthalic anhydride-co-1,3-phenylenediamine), etc. In further embodiments, the sequestration polymer may be a polybenzimidazoles. In additional embodiments, the sequestration polymer may be an amphiphilic copolymer (e.g., a PMMA-polyethylene glycol (PMMA-PEG) copolymer). For example, the sequestration polymer in reactor 380 may be an amphiphilic copolymer, or other polymer, with carbonyl moieties that is miscible with polyvinylidene difluoride (PVDF).

As discussed above, combinations of reactors 206 including at least one reactor selected from reactors 310-380 as examples of reactors 206-1, 206-2, and/or 206-N may be used to defluorinate mixtures of fluorinated compounds from source 203 in process 100. For example, reactor 360 may partially defluorinate a mixture via hydrogenation at operation 130, and reactor 310 may carry out additional defluorination with an alkali metal catalyst on oxide support at operation 150. In another example, reactor 370 may partially defluorinate a mixture via defluorohydrogenation at operation 130, and reactor 360 may carry out additional defluorination via palladium-catalyzed defluorohydrogenation at operation 150.

The descriptions of the various embodiments of the present disclosure have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A process, comprising:
receiving, at a first reactor from a set of reactors, a mixture of fluorinated compounds;
partially defluorinating, by a first defluorinating species in the first reactor, the mixture of fluorinated compounds, wherein the first defluorinating species is a solid supported species; and
substantially defluorinating, by at least one additional defluorinating species in at least one additional reactor from the set of reactors, the partially defluorinated mixture of fluorinated compounds.

2. The process of claim 1, wherein the partially defluorinating the mixture of fluorinated compounds comprises partially defluorinating a first species of fluorinated compound in the mixture of fluorinated compounds.

3. The process of claim 2, wherein the substantially defluorinating the mixture of fluorinated compounds comprises completely defluorinating the partially defluorinated first species of fluorinated compound.

4. The process of claim 1, wherein the partially defluorinating the mixture of fluorinated compounds comprises completely defluorinating a first species of fluorinated compound in the mixture of fluorinated compounds.

5. The process of claim 1, wherein the first defluorinating species is a photocatalyst.

6. The process of claim 5, wherein the photocatalyst is selected from the group consisting of [4,4′-bis(1,1-dimethylethyl)-2,2′-bipyridine-N1,N1′]bis[2-(2-pyridinyl-N)phenyl-C]iridium(III) hexafluorophosphate, tris(2,2′-bipyridine)ruthenium(II) hexafluorophosphate, N-phenylphenothiazine, 1,2,3,5-tetrakis(carbazol-9-yl)-4,6-dicyanobenzene, and [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate.

7. The process of claim 1, wherein the first defluorinating species is an alkali metal absorbed by an oxide.

8. The process of claim 1, wherein the first defluorinating species is selected from the group consisting of a palladium hydrogenation catalyst, a photocatalyst, a sequestration polymer, and an alkali metal.

9. The process of claim 8, wherein:
the at least one additional reactor is a photoreactor; and
the at least one additional defluorinating species comprises hydrated electrons.

10. The process of claim 1, wherein the first defluorinating species can defluorinate at least one species of fluorinated compound selected from the group consisting of trifluoromethyl arenes, aromatic fluorides, difluoroalkenes, per- and polyfluoroalkyl substances, and sulfur hexafluoride.

11. A system, comprising:
a first reactor, wherein the first reactor is configured to:
receive a mixture of fluorinated compounds; and
partially defluorinate, by a first defluorinating species, the mixture of fluorinated compounds, wherein the first defluorinating species is a solid supported species; and
at least one additional reactor, wherein the at least one additional reactor is configured to substantially defluorinate, by at least one additional defluorinating species, the partially defluorinated mixture of fluorinated compounds.

12. The system of claim 11, wherein the partially defluorinating the mixture of fluorinated compounds comprises partially defluorinating a first species of fluorinated compound in the mixture of fluorinated compounds.

13. The system of claim 12, wherein the substantially defluorinating the mixture of fluorinated compounds comprises completely defluorinating the partially defluorinated first species of fluorinated compound.

14. The system of claim 11, wherein the partially defluorinating the mixture of fluorinated compounds comprises completely defluorinating a first species of fluorinated compound in the mixture of fluorinated compounds.

15. The system of claim 11, wherein the first defluorinating species is a photocatalyst.

16. The system of claim 15, wherein the photocatalyst is selected from the group consisting of [4,4′-bis(1,1-dimethylethyl)-2,2′-bipyridine-N1,N1′]bis[2-(2-pyridinyl-N)phenyl-C]iridium(III) hexafluorophosphate, tris(2,2′-bipyridine)ruthenium(II) hexafluorophosphate, N-phenylphenothiazine, 1,2,3,5-tetrakis(carbazol-9-yl)-4,6-dicyanobenzene, and [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate.

17. The system of claim 11, wherein the first defluorinating species is selected from the group consisting of a palladium hydrogenation catalyst, a photocatalyst, a sequestration polymer, and an alkali metal.

18. The system of claim 17, wherein:
the at least one additional reactor is a photoreactor; and
the at least one additional defluorinating species comprises hydrated electrons.

19. The system of claim 11, wherein the first defluorinating species can defluorinate at least one species of fluorinated compound selected from the group consisting of trifluoromethyl arenes, aromatic fluorides, difluoroalkenes, per- and polyfluoroalkyl substances, and sulfur hexafluoride.

20. A system, comprising:
a set of reactors configured to operate in sequence to substantially defluorinate a mixture of fluorinated compounds, wherein the operating in sequence comprises:
receiving, at a first reactor from the set of reactors, the mixture of fluorinated compounds;
defluorinating, by a first defluorinating species in the first reactor, a first species of fluorinated compound in the mixture of fluorinated compounds; and
defluorinating, by a second defluorinating species in a second reactor from the set of reactors, a second species in the mixture of fluorinated compounds.

21. A process, comprising:
substantially defluorinating, by a set of reactors configured to operate in sequence, a mixture of fluorinated compounds, wherein the substantially defluorinating comprises:
receiving, at a first reactor from the set of reactors, the mixture of fluorinated compounds;
defluorinating, by a first defluorinating species in the first reactor, a first species of fluorinated compound in the mixture of fluorinated compounds; and
defluorinating, by a second defluorinating species in a second reactor from the set of reactors, a second species in the mixture of fluorinated compounds.

22. A system, comprising:
a set of reactors configured to operate in sequence to substantially defluorinate a mixture of fluorinated compounds, wherein:
the set of reactors comprises a reactor containing a first defluorinating species on a solid support;
the first defluorinating species is an alkali metal; and
the solid support is an oxide.

23. The system of claim 22, wherein the reactor also contains a solution of a redox mediator.

24. The system of claim 23, wherein the reactor also contains bare silica.

25. The system of claim 22, wherein:
the set of reactors further comprises a next reactor containing a second defluorinating species on a second solid support; and
the second defluorinating species is selected from the group consisting of a hydrogenation catalyst, a photocatalyst, and a sequestration polymer.
